(19) United States
(12) Plant Patent Application Publication
(10) Pub. No.: US 2004/0216198 P1
(43) Pub. Date: Oct. 28, 2004

(54) Sedum purpureum 'Pink Chablis'

(76) Inventor: Christopher B. Howe, Nunica, MI (US)

Correspondence Address: David S MacKenzie, Hortech Inc, P O Box 533, Spring Lake, MI, 49456, US

(21) Appl. No.: 09/514,930

(22) Filed: Oct. 15, 2001

(51) Int. Cl. A01H 5/00

(52) U.S. Cl. PLT/263

(57) ABSTRACT

A showy sedum variety named ‘Pink Chablis’, characterized by white and blue-green variegated leaves, compact habit, and pink fallborne flowers.

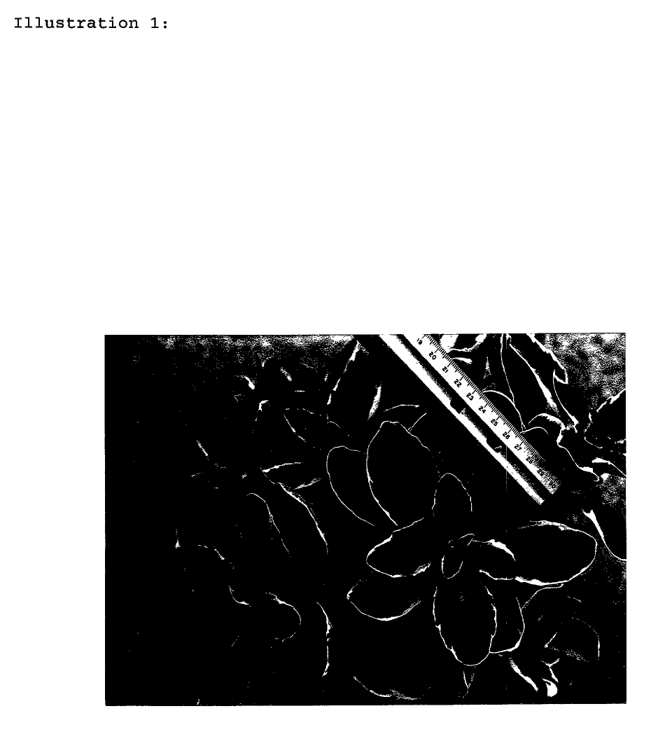

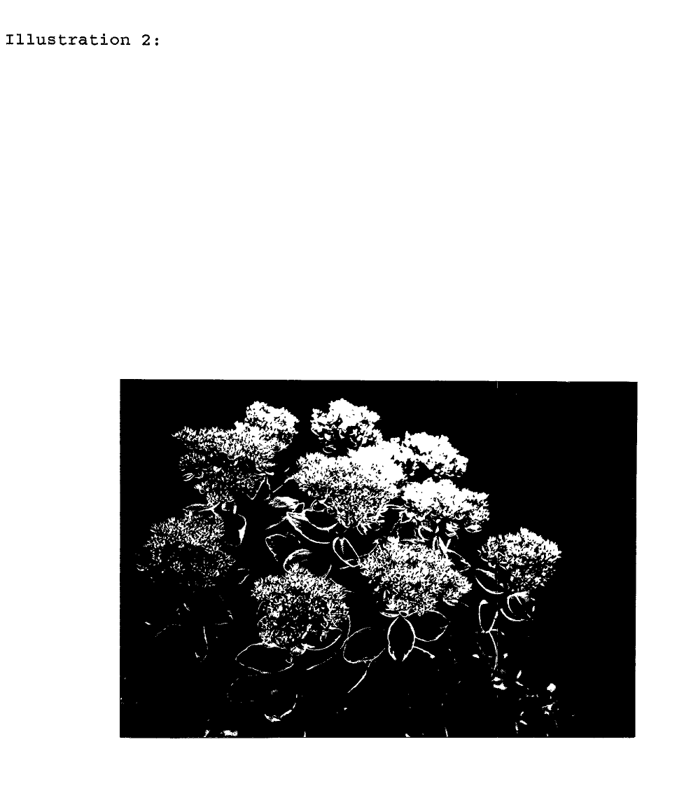

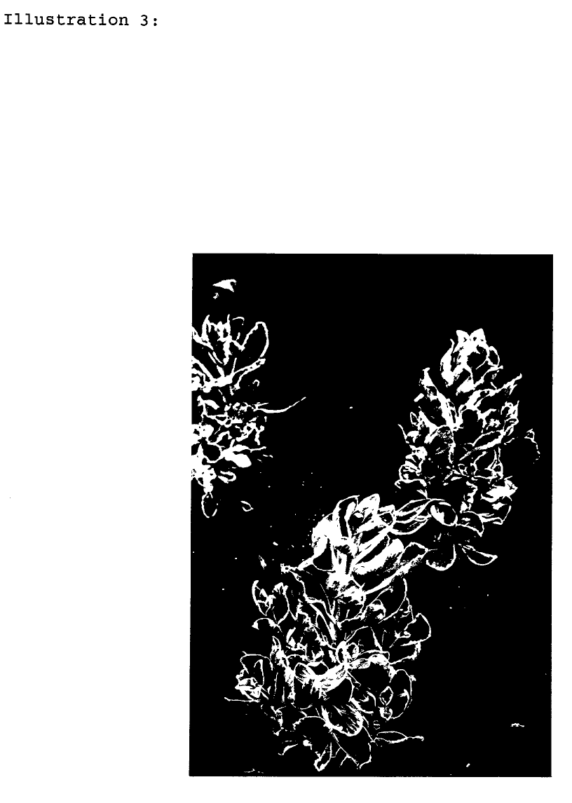

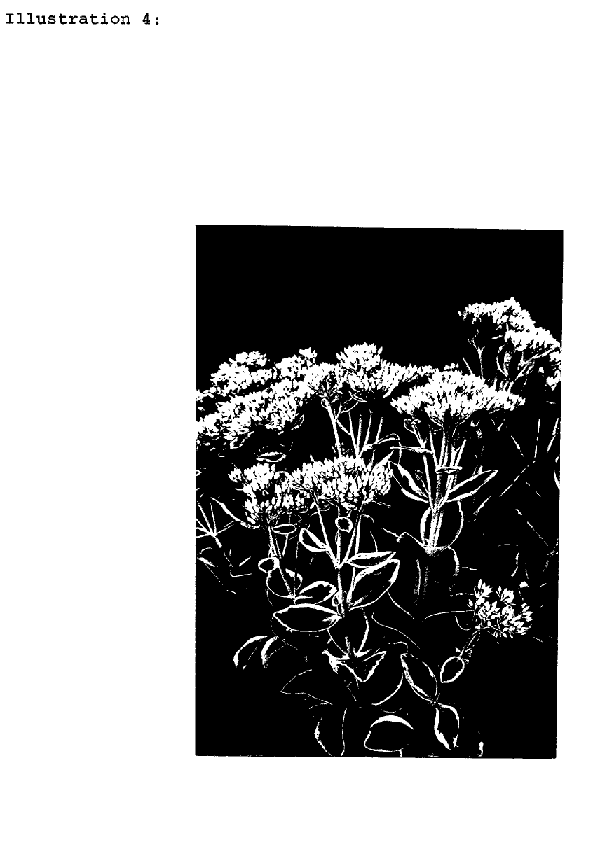

The present invention comprises a new and distinct cultivar of Sedum purpureum, hereafter referred to by the cultivar name ‘Pink Chablis’

‘Pink Chablis’ is a new and distinct variety of showy stonecrop, a succulent clump forming cold hardy perennial from Asia. ‘Pink Chablis’ is unique and outstanding for its broad, horizontally carried, flattened leaves with white-variegated margins.

In addition, ‘Pink Chablis’ bears broad rounded clusters of white budded, pink fallborne flowers.

On Feb. 28, 1996 international registration as an herbaceous (in this case succulent) plant cultivar was applied for. The international registrar at the International Plant Union in Freising Germany approved the application for registration on Mar. 22, 1996.

‘Pink Chablis’ originated as a branch sport (mutation) from the species Sedum purpureum and is similar to the parent species in the morphology of its flowers stems and foliage. The first successful asexual reproduction of ‘Pink Chablis’ was carried out by its discoverer, Chris Howe, employee of Hortech. This propagation took place under controlled conditions in Nunica, Mich., on Jun. 15, 1995.

The sport was excised, treated with 3000 ppm IBA, and rooted under intermittent mist over a period of two weeks. One cutting rooted and was transplanted for evaluation.

‘Pink Chablis’ has not been observed under all possible environmental conditions, and the phenotype may vary significantly with variations in environment, such as temperature, light intensity and day length. The following observations, measurements, and comparisons describe this plant as grown in Nunica, Mich. when container grown (outdoors without cover) in conditions that approximate those generally used in commercial practice.

### BRIEF DESCRIPTION OF THE VARIOUS VIEWS OF THE DRAWINGS

The accompanying photographic illustrations show typical foliage of the cultivar and the closest known comparison plant (the cultivar Sedum ‘Frosty Morn’) with color being as accurate as possible with renditions of this type. The photographs were taken at 1:00 P.M. July 1st (foliage depictions) and Sep. 1st (floral depictions) 1999 under cloudy conditions, in Nunica, Mich. The film used was Fujifilm ISO 200 exposed for 1/30 second at F8 using no filters. Illustrations 1-2 are perspective views of Sedum purpureum ‘Pink Chablis’. Illustrations 3 to 4 are perspective views of Sedum purpureum ‘Frosty Morn’.

The following traits have been repeatedly observed and are determined to be the basic characteristics of ‘Pink Chablis’, which in combination distinguish this showy stonecrop as a new and distinct cultivar. The color codes correspond to The Royal Horticultural Society Color Chart.

1. Deciduous foliage is simple, opposite, and sometimes whorled. In youth, the foliage is obovate in outline, 1 inch long, ½ inch wide, serrate above the middle, hairless, tacky to the touch, and colored light-green 129-C. Leaf margins are colored green white 157-A, 1/16 inch to 3/16 inch wide. Petioles at this stage are wiry, 1/16 inch wide, colored light-green 129-C.

2. In maturity, the foliage is obovate in outline, hairless, 3-3½ inches long, 1½-2 inches wide colored green 129-A with slender irregular areas of lighter green 129-C. Leaf margins are colored green white 157-A. ⅛ inch to ⅜ inch wide. Petioles at this stage are thickened, ¼ inch wide, colored green 128-D.

3. The midvein is light green 130-C and is only slightly raised above the blade. It gives rise to smaller and equally visible secondary veins of the same color.

4. Stems are herbaceous and arise vertically from a clump. Typically they are erectly oriented. Stems reach 14 to 18 inches in length, ¼ to ½ inch wide. The stems are round in cross section and range from ¼ inch to ⅜ inch wide. In youth and in maturity they are colored green 128-D.

5. Internodes range in length from 1 to 1½ inches.

6. Bisexual flowers are pink, 64-C, and borne in August, lasting through September. Individual flowers are ¼ inch wide and 5 petaled. The panicles are 5 to 6½ inches wide, dense, with 20 to 30 forked branches. Flowers exude a mild sweet fragrance. The flowers are with five red pistils ¼ to ⅜ inch in length, five stamens ¼ to ⅜ inch in length and 5 white sepals 1/32 inch wide and 1/16 inch long.

7. Aggregate fruit is 1/16 inch to 2/16 inch in length, 1/32 to 2/32 inch wide at the base rising to a pointed tip.

Other cultivars of Sedum purpureum include ‘Frosty Morn’, which differs in having more slender curled leaves orientated at 45 degree angles from the stems. The flowers of ‘Frosty Morn’ are less dense and uniform. The panicles are 3 to 4 inches wide with 10-20 forked branches.

1. The present invention comprises a new and distinct cultivar of Sedum purpureum (referred to by the cultivar name ‘Pink Chablis’) which is an herbaceous perennial from Asia unique and outstanding for its variegated blue-green and white foliage, white flower buds, pink flowers, and compact habit.
